(19) United States
(12) Patent Application Publication
(10) Pub. No.: US 2016/0193340 A1
(43) Pub. Date: Jul. 7, 2016

(54) NOVEL USES OF ALKYL POLYGLYCOSIDES FOR SOLUBILISING VITAMIN E IN WATER; COMPOSITIONS COMPRISING SAME

(71) Applicant: SOCIETE D'EXPLOITATION DE PRODUITS POUR LES INDUSTRIES CHIMIQUES SEPPIC, Paris Cedex 07 (FR)

(72) Inventors: Marie-Laure BRETON, CASTRES (FR); Sebastien KERVERDO, VINCENNES (FR); Stephanie GARCEL, CASTRES (FR); Jerome GUILBOT, CASTRES (FR); Herve ROLLAND, CASTRES (FR)

(21) Appl. No.: 14/909,336

(22) PCT Filed: Jul. 23, 2014
(86) PCT No.: PCT/FR2014/051905

§ 371 Date: Feb. 1, 2016

(30) Foreign Application Priority Data
Jul. 31, 2013 (FR) 1357591

(51) Int. Cl. A61K 47/26; A61K 31/355; A61Q 5/00; A61Q 19/10; A61Q 5/02; A61Q 19/08; A61K 8/67; A61K 8/60

(52) U.S. Cl. CPC A61K 47/26; A61K 8/678; A61K 31/355; A61K 8/604; A61Q 19/10; A61Q 5/02; A61Q 19/08; A61Q 5/00; A61K 2800/49

(57) ABSTRACT

A composition represented by formula (I): R—O-(G)ₚ-H (I) in which R represents a radical including 6 to 8 carbon atoms, G represents the residue of a reducing sugar and p represents a decimal number greater than or equal to 1.05 and less than or equal to 5, and the composition is a mixture of compounds represented by formulae (I1), (I2), (I3), (I4) and (I5): R—O-(G)1-H(I1) R—O-(G)2-H(I2) R—O-(G)3-H(I3) R—O-(G)4-H(I4) R—O-(G)5-H(I5), in respective molar proportions a1, a2, a3, a4 and a5, such that: a1+a2+a3+a4+a5=1. The use of the composition as a solubilising agent in an aqueous composition of at least one compound (A) chosen from α-tocopherol, β-tocopherol, γ-tocopherol, δ-tocopherol, α-tocopherol acetate, β-tocopherol acetate, γ-tocopherol acetate, δ-tocopherol acetate, α-tocotrienol, β-tocotrienol, γ-tocotrienol and δ-tocotrienol. Also, a composition (C2) and its use in methods for preventing the unattractive effects of ageing, for cleaning the skin and hair, and for therapeutic treatment.

The present invention relates to the use of an alkyl polyglycoside or of a mixture of alkyl polyglycosides of which the alkyl chain comprises from 6 to 8 carbon atoms for solubilizing vitamin E, which is even in the form of a tocopherol, or in the form of a tocotrienol, in aqueous compositions. The invention also relates to aqueous compositions comprising such alkyl polyglycosides and vitamin E, to the uses thereof for cleansing, for protecting and/or for caring for the skin, the hair, the scalp and the mucous membranes, and also to the uses thereof for pharmaceutical and food applications.

Vitamin E is the most important liposoluble antioxidant present in the mammalian body. It is introduced into the mammalian body by the diet, for example by ingesting fresh vegetables, vegetable oils, cereals, nuts, certain meats and certain dairy products. The term “vitamin E” denotes eight natural forms:

α-tocopherol or (2R)-2,5,7,8-tetramethyl-2-[(4R,8R)-4,8,12-trimethyltridecyl]-3,4-dihydrochromen-6-ol,

β-tocopherol or (2R)-2,5,8-trimethyl-2-[(4R,8R)-4,8,12-trimethyltridecyl]-3,4-di-hydrochromen-6-ol,

γ-tocopherol or (2R)-2,7,8-trimethyl-2-[(4R,8R)-4,8,12-trimethyltridecyl]-3,4-di-hydrochromen-6-ol,

δ-tocopherol or (2R)-2,8-dimethyl-2-[(4R,8R)-4,8,12-trimethyltridecyl]-3,4-di-hydrochromen-6-ol,

α-tocotrienol or (2R)-2,5,7,8-tetramethyl-2-[(3E,7E)-4,8,12-trimethyltrideca-3,7,11-trienyl]-3,4-dihydrochromen-6-ol,

β-tocotrienol or (2R)-2,5,8-trimethyl-2-[(3E,7E)-4,8,12-trimethyltrideca-3,7,11-trienyl]-3,4-dihydrochromen-6-ol,

γ-tocotrienol or (2R)-2,7,8-trimethyl-2-[(3E,7E)-4,8,12-trimethyltrideca-3,7,11-trienyl]-3,4-dihydrochromen-6-ol and

δ-tocotrienol or (2R)-2,8-dimethyl-2-[(3E,7E)-4,8,12-trimethyltrideca-3,7,11-trienyl]-3,4-dihydrochromen-6-ol.

Tocopheryl acetates, such as a-tocopheryl acetate, β-tocopheryl acetate, γ-tocopheryl acetate or δ-tocopheryl acetate, are synthetic compounds obtained from the corresponding tocopherols which also have advantageous antioxidant properties. Consequently, vitamin E and vitamin E acetate are commonly used as antioxidants used for the preparation of cosmetic, pharmaceutical and food compositions.

Vitamin E is, for example, used as a preservative in food compositions in order to prevent foods from turning rancid. More generally, vitamin E is combined with unsaturated fatty phases in order to limit oxidation phenomena.

Vitamin E is particularly abundant in the stratum corneum, which is the first layer of the skin to experience external oxidative stresses. Indeed, during chronological or actinic aging thereof, the skin is subjected to various oxidative stresses which are either physiological or are the result of the action of external agents, such as ultraviolet radiation, atmospheric pollution or the consumption of tobacco. These oxidative stresses result from the skin cells being attacked by reactive free-radical species such as the superoxide radical, the hydroxyl radical, singlet oxygen or the peroxynitrite radical. They cause tissue and cell modifications, or even cell mortality, and therefore contribute to skin aging, which can result in collagen degradation, resulting in stiffening of the skin. Vitamin E, because of the nature of its chemical structure, plays a protective role against skin aging, more particularly caused by external free-radical oxidizing agents. Vitamin E plays more particularly a photoprotective role on the skin against UV radiation, and in particular against acute damage caused by inflammation (erythema, sunburn) and by hyperpigmentation (tanning) induced by prolonged exposure to UV radiation, or against chronic damage of skin cancer induced by UV radiation [Burke, K. E. et al., “The effects of topical and oral vitamin E on pigmentation and skin cancer induced by ultraviolet irradiation in Skh:2 hairless mice”; Nutr. Cancer 38:87-97, 2000; Roshchupkin, D. et al., “Inhibition of ultraviolet light-induced erythema by antioxidants.”, Arch Dermatol. Res. 266:91-94, 1979; Marenus, K. et al., “The use of antioxidants in providing protection from chronic suberythemal UV-8 exposure”, 16th IFSCC Conference, 1 Oct. 1990:24-34; Gerrish, K. et al. “Prevention of photocarcinogenesis by dietary vitamin E.” Nutr Cancer 19:125-133, 1993].

The action of these oxidative stresses on the skin generates a decrease in the amount of vitamin E in the stratum corneum, thus requiring a balanced diet and/or a provision of vitamin E supplementary to the diet, in the form of the oral, parenteral or topical administration of compositions comprising vitamin E.

The liposoluble nature of vitamin E allows it to be easily incorporated into the fatty phases present in the various galenical forms, such as water-in-oil emulsions or oil-in-water emulsions, and therefore into topically administered cosmetic and pharmaceutical formulations.

However, some specific fields of use of vitamin E can require it to be used in aqueous media, for example:

for preparing cosmetic compositions for cleansing and/or for protecting and/or caring for the hair and/or the scalp;

for preparing aqueous gels and/or aqueous sera intended for the protection and/or caring for the skin and/or the mucous membranes, for example anti-aging sera and/or gels; or

for preparing aqueous food compositions such as drinks, milk drinks, energy drinks or dairy products.

There are several ways known in the prior art to solve the problem of solubilizing vitamin E in aqueous phases. The international application published under number WO 91/11189 describes cosmetic compositions prepared from a suspension of vesicles comprising a disodium salt of tocopheryl phosphate. The international application published under number WO 2003/094882 describes a cosmetic composition comprising a sodium salt of tocopheryl phosphate, which is in the form of an aqueous solution of low concentration or of a dispersion. The international application published under number WO 2005/102267 describes a composition of which the aqueous phase comprises tocopheryl glycinate. However, each of the elements of the prior art mentioned above teaches that the water-solubility of the tocopheryl salt, although improved compared with that of tocopherol, is not however sufficient to integrate greater weight proportions of vitamin E into the final galenical form.

U.S. Pat. No. 5,990,180 describes the use of a polyglycerol ester of a fatty acid comprising from 12 to 14 carbon atoms for solubilizing vitamin E. The international application published under number WO 2008/030312 discloses the use of salts of a monoester of a dicarboxylic acid and of a fatty alcohol comprising from 8 to 22 carbon atoms for solubilizing vitamin E.

The European patent application published under number EP 0 106 100 A2 discloses the use of saponins as an agent for solubilizing vitamin E, and more particularly saponins originating from plant extracts, such as quillaia saponin or tea saponin.

The French patent application published under number FR 2 816 517 A1 discloses the preparation of alkyl polyglycosides from fusel oil and from at least one reducing sugar in an acidic catalytic medium, and the use of said alkyl polyglycosides on fusel oil as an agent for solubilizing oily or liposoluble ingredients. Fusel oils are mixtures of alcohols comprising in particular short-chain alcohols such as ethanol, 1-propanol, 2-propanol, 1-methylpropanol, 1-butanol, 2-methylbutanol and 3-methylbutanol, which require the implementation of anti-explosion safety measures when they are used industrially.

The international application published under number WO 96/33255A1 describes antifoam compositions comprising a particular alkyl polyglucoside, the alkyl chain of which consists of the 2-ethylhexyl radical, and defoaming nonionic surfactants chosen from those comprising one or more groups chosen from monoethoxylated or polyethoxylated groups, and monopropoxylated or polypropoxylated groups. It is taught therein that 2-ethylhexyl-chain alkyl polyglucosides are more effective than hexyl-chain alkyl polyglycosides for solubilizing defoaming nonionic surfactants.

The international application published under number WO 99/21948A1 discloses compositions which are clear and stable at high alkaline concentrations, the foaming properties of which are controlled, and which contain a large amount of nonionic surfactants based on alkylene oxide and a hexyl glycoside as hydrotropic agent. It is taught therein that hexyl glycosides and more particularly n-hexyl polyglucoside make it possible to solubilize nonionic surfactants in strongly alkaline media.

The international application published under number WO 2012/069730 A1 discloses compositions which are clear and stable at high concentrations both of alkaline species and of electrolytic species, comprising a large amount of nonionic surfactants based on alkylene oxide and n-heptyl polyglucoside as hydrotropic agent.

Nothing in the prior art teaches that the alkyl polyglycosides prepared from n-hexanol, or from n-heptanol, or from 2-ethylhexanol, and known for their hydrotropic properties, namely solubilizing in water surfactants which have low solubility in strongly alkaline, or acid, or electrolytic media, can constitute agents for solubilizing vitamin E.

Consequently, according to a first aspect, a subject of the invention is the use of a composition represented by formula (I):

R—O-(G)ₚ-H (I)

in which R represents a radical chosen from n-hexyl, n-heptyl or 2-ethylhexyl radicals, G represents the residue of a reducing sugar and p represents a decimal number greater than or equal to 1.05 and less than or equal to 5, said composition of formula (I) consisting of a mixture of compounds represented by formulae (I_{I}), (I₂), (I₃), (I₄) and (I₅):

R—O-(G)₁-H (I₁)

R—O-(G)₂-H (I₂)

R—O-(G)₃-H (I₃)

R—O-(G)₄-H (I₄)

R—O-(G)₅-H (I₅),

in the respective molar proportions a₁, a₂, a₃, a₄ and a₅, such that:

the sum a₁+a₂+a₃+a₄+a₅ is equal to 1 and that

each of the proportions a₁, a₂, a₃, a₄ and a₅ is greater than or equal to zero and less than or equal to one,

as an agent for solubilizing in an aqueous composition at least one compound (A) chosen from α-tocopherol, le β-tocopherol, γ-tocopherol, δ-tocopherol, α-tocopheryl acetate, β-tocopheryl acetate, γ-tocopheryl acetate, δ-tocopheryl acetate, α-tocotrienol, β-tocotrienol, γ-tocotrienol and δ-tocotrienol.

The term “reducing sugar” denotes, in formula (I), the saccharide derivatives which do not exhibit in their structures any glycosidic bond established between an anomeric carbon and the oxygen of an acetal group such as they are defined in the reference handbook: “Biochemistry”, Daniel Voet/Judith G. Voet, p. 250, John Wyley & Sons, 1990. The oligomeric structure (G)p can be in any of the isomer forms, and which is an optical isomer, a geometric isomer or a positional isomer; it can also represent a mixture of isomers.

The terms “α-tocopherol”, “β-tocopherol”, “γ-tocopherol”, “δ-tocopherol”, “α-tocotrienol”, “β-tocotrienol”, “γ-tocotrienol” and “δ-tocotrienol” denote either the diastereoisomerically pure forms of each of said compounds as stated in the preamble of the present description, or the racemates of said compounds, for example DL-α-tocopherol.

In formula (I) as defined above, the R—O group is bonded to G by the anomeric carbon of the saccharide residue, so as to form an acetal function.

According to one particular aspect, a subject of the invention is the use as defined above, characterized in that, in formula (I), p represents a decimal number greater than or equal to 1.05 and less than or equal to 2.5.

According to another particular aspect, a subject of the invention is the use as defined above, for which, in the definition of the compounds of formula (I), G represents the residue of a reducing sugar chosen from glucose, dextrose, sucrose, fructose, idose, gulose, galactose, maltose, isomaltose, maltotriose, lactose, cellobiose, mannose, ribose, xylose, arabinose, lyxose, allose, altrose, dextran or tallose, more particularly a residue of a reducing sugar chosen from the residues of glucose, of xylose and of arabinose.

According to another particular aspect, R represents the n-hexyl radical; according to another particular aspect, R represents the n-heptyl radical; and according to another final particular aspect, R represents the 2-ethylhexyl radical.

According to another particular aspect, a subject of the invention is the use as defined above, for which, in formula (I), R represents a radical chosen from the n-hexyl, n-heptyl and 2-ethylhexyl radicals, G represents the residue of a reducing sugar chosen from glucose, xylose and arabinose, and p represents a decimal number greater than or equal to 1.05 and less than or equal to 2.5; for example, the use as defined above, for which, in formula (I), R represents the n-hexyl radical, G represents the residue of glucose and p represents a decimal number greater than or equal to 1.05 and less than or equal to 2.5; or the use as defined above, for which, in formula (I), R represents the n-heptyl radical, G represents the residue of glucose and p represents a decimal number greater than or equal to 1.05 and less than or equal to 2.5; or the use as defined above, for which, in formula (I), R represents the 2-ethylhexyl radical, G represents the residue of glucose and p represents a decimal number greater than or equal to 1.05 and less than or equal to 2.5.

According to another particular aspect, a subject of the invention is the use as defined above, for which the compound (A) is chosen from α-tocopherol, β-tocopherol, γ-tocopheol, δ-tocopherol, α-tocopheryl acetate, β-tocopheryl acetate, γ-tocopheryl acetate and δ-tocopheryl acetate and more particularly the compound (A) is α-tocopherol or α-tocopheryl acetate, for example the use as defined above, for which the compound (A) is α-tocopherol or the use as defined above, for which the compound (A) is α-tocopheryl acetate.

According to another particular aspect, a subject of the invention is either the use as defined above, for which, in formula (I), R represents the n-hexyl radical, G represents the residue of glucose and p represents a decimal number greater than or equal to 1.05 and less than or equal to 2.5 and the compound (A) is α-tocopherol or α-tocopheryl acetate; or the use as defined above, for which, in formula (I), R represents the n-heptyl radical, G represents the residue of glucose and p represents a decimal number greater than or equal to 1.05 and less than or equal to 2.5 and the compound (A) is α-tocopherol or α-tocopheryl acetate; or the use as defined above, for which, in formula (I), R represents the 2-ethylhexyl radical, G represents the residue of glucose and p represents a decimal number greater than or equal to 1.05 and less than or equal to 2.5 and the compound (A) is α-tocopherol or α-tocopheryl acetate.

According to another particular aspect, a subject of the invention is the use as defined above, characterized in that the weight ratio between the compound (A) and the composition represented by formula (I) is greater than or equal to 1/10 and less than or equal to 6.5/10, more particularly greater than or equal to 2/10 and less than or equal to 6.5/10, and even more particularly greater than or equal to 2.5/10 and less than or equal to 6.5/10.

A subject of the invention is also a composition (C₂) comprising, for 100% of its weight:

a) from 0.05% to 12.0% by weight of at least one composition represented by formula (I) as defined above;

b) from 0.01 to 5.0% by weight of at least one compound (A) chosen from α-tocopherol, β-tocopherol, γ-tocopherol, δ-tocopherol, α-tocopheryl acetate, β-tocopheryl acetate, γ-tocopheryl acetate, δ-tocopheryl acetate, α-tocotrienol, β-tocotrienol, γ-tocotrienol or δ-tocotrienol, and

c) from 83.0% to 99.94% by weight of water.

According to one particular aspect, a subject of the invention is a composition (C₂) as defined above, for which, in formula (I), p represents a decimal number greater than or equal to 1.05 and less than or equal to 2.5.

According to another particular aspect, a subject of the invention is a composition (C₂) as defined above, for which, in formula (I), G represents the residue of a reducing sugar chosen from the residues of glucose, dextrose, sucrose, fructose, idose, gulose, galactose, maltose, isomaltose, maltotriose, lactose, cellobiose, mannose, ribose, xylose, arabinose, lyxose, allose, altrose, dextran or tallose; G representing more particularly the residue of a reducing sugar chosen from the residues of glucose, of xylose and of arabinose.

According to another particular aspect, a subject of the invention is a composition (C₂) as defined above, characterized in that, in formula (I), the R radical represents a radical chosen from the n-hexyl, n-heptyl and 2-ethylhexyl radicals, for example a composition (C₂) as defined above, for which, in formula (I), R represents the n-hexyl radical; or a composition (C₂) as defined above, for which, in formula (I), R represents the n-heptyl radical or a composition (C₂) as defined above, for which, in formula (I), R represents the 2-ethylhexyl radical.

According to another particular aspect, a subject of the invention is a composition (C₂) as defined above, for which, in formula (I), R represents a radical chosen from the n-hexyl, n-heptyl and 2-ethylhexyl radicals, G represents the residue of a reducing sugar chosen from the residues of glucose, xylose and arabinose, and p represents a decimal number greater than or equal to 1.05 and less than or equal to 2.5; for example, a composition (C₂) as defined above, for which, in formula (I), R represents the n-hexyl radical, G represents the residue of glucose and p represents a decimal number greater than or equal to 1.05 and less than or equal to 2.5; or a composition (C₂) as defined above, for which, in formula (I), R represents the n-heptyl radical, G represents the residue of glucose and p represents a decimal number greater than or equal to 1.05 and less than or equal to 2.5; or a composition (C₂) as defined above, for which, in formula (I), R represents the 2-ethylhexyl radical, G represents the residue of glucose and p represents a decimal number greater than or equal to 1.05 and less than or equal to 2.5.

According to another particular aspect, a subject of the invention is a composition (C₂) as defined above, in which the compound (A) is chosen from α-tocopherol, β-tocopherol, γ-tocopherol, δ-tocopherol, α-tocopheryl acetate, β-tocopheryl acetate, γ-tocopheryl acetate, δ-tocopheryl acetate; and more particularly a composition (C₂) as defined above, characterized in that the compound (A) is α-tocopherol or α-tocopheryl acetate; for example, a composition (C₂) as defined above, in which the compound (A) is α-tocopherol or a composition (C₂) as defined above, in which the compound (A) is α-tocopheryl acetate.

According to another particular aspect, a subject of the invention is a composition (C₂) as defined above, for which, in formula (I), R represents the n-hexyl radical, G represents the residue of glucose and p represents a decimal number greater than or equal to 1.05 and less than or equal to 2.5, and in which the compound (A) is α-tocopherol or α-tocopheryl acetate; or else a composition (C₂) as defined above, for which, in formula (I), R represents the n-heptyl radical, G represents the residue of glucose and p represents a decimal number greater than or equal to 1.05 and less than or equal to 2.5, and in which the compound (A) is α-tocopherol or α-tocopheryl acetate; or else a composition (C₂) as defined above, for which, in formula (I), R represents the 2-ethylhexyl radical, G represents the residue of glucose and p represents a decimal number greater than or equal to 1.05 and less than or equal to 2.5, and in which the compound (A) is α-tocopherol or α-tocopheryl acetate.

According to another particular aspect, a subject of the invention is a composition (C₂) as defined above, in which the weight ratio between the compound (A) and the composition represented by formula (I) is greater than or equal to 1/10 and less than or equal to 6.5/10, more particularly greater than or equal to 2/10 and less than or equal to 6.5/10 and even more particularly greater than or equal to 2.5/10 and less than or equal to 6.5/10.

The composition (C₂) according to the invention is prepared by mixing its constituents, with mechanical stirring, at a stirring speed of between 50 revolutions/minute and 500 revolutions/minute, more particularly between 50 revolutions/minute and 100 revolutions/minute, at a temperature of between 20° C. and 80° C., more particularly between 20° C. and 60° C., and even more particularly between 20° C. and 45° C.

A subject of the invention is also the use of the composition (C₂) as defined above, for preventing the unattractive effects of aging of the skin, the hair, the scalp or the mucous membranes and more particularly those induced by oxidative damage originating from free radicals, in particular those generated by solar radiation, such as ultraviolet solar radiation.

The term “aging of the skin” is intended to mean a progressive physiological phenomenon which manifests itself through a yellowish or translucent complexion associated with thinning of the dermis, reduction of the adipose pannicules, wrinkles and/or a reduction in skin tone.

The term “aging of the hair” is intended to mean a progressive physiological phenomenon which manifests itself through cuticle damage and results in a rougher surface condition of the hair, in fiber damage, which can then cause breaks and/or in a loss of sheen of the hair.

A subject of the invention is also the use of the composition (C₂) as defined above, for preparing a composition intended for cleansing the skin, the hair, the scalp or the mucous membranes.

The expression “cleansing of the skin, the hair, the scalp or the mucous membranes” denotes any action intended to make it possible to remove soiling present on the skin, the hair, the scalp or the mucous membranes of human beings or of animals. Examples of soiling present on the skin, the hair, the scalp or the mucous membranes of human beings or of animals are dust, earth, sebaceous secretions, sweat, dandruff, dead cells, microorganisms or various chemical substances such as the residues of makeup and care compositions for the skin, the hair, the scalp or the mucous membranes.

When it is used for cosmetic purposes, namely for modifying the external appearance of the skin, the hair, the scalp or the mucous membranes, for example for preventing unattractive effects of aging, or for cleansing, the composition (C₂) which is the subject of the present invention is more particularly administered topically.

The expression “topical” means that the composition (C₂) according to the invention is used by application to the skin, the hair, the scalp or the mucous membranes, whether it is a direct application or an indirect application, when for example the composition (C₂) according to the invention is incorporated into a support intended to be brought into contact with the skin (paper, wipe, textile, transdermal device, etc.).

The topical composition (C₂) according to the invention may be packaged in a bottle, and also in pressurized form in an aerosol device or in a device of “pump-action bottle” type, in a device equipped with a perforated wall, for example a grid, or in a device equipped with a ball applicator (termed “roll-on”).

The topical composition (C₂) according to the invention generally comprises chemical additives normally used in the field of formulations for topical use, which are in particular cosmetic, dermocosmetic, pharmaceutical or dermopharmaceutical, such as foaming and/or detergent surfactants, thickening and/or gelling surfactants, thickeners and/or gelling agents, stabilizers, film-forming compounds, solvents and cosolvents, hydrotropic agents, plasticizers, emulsifiers and coemulsifiers, opacifiers, nacreous agents, overfatting agents, sequestering agents, chelating agents, oils, waxes, antioxidants, fragrances, essential oils, preservatives, conditioning agents, deodorants, whitening agents intended for bleaching body hair and the skin, active ingredients intended to provide a treating and/or protective action with respect to the skin or the hair, sunscreens, mineral fillers or pigments, particles which provide a visual effect or which are intended for encapsulating active agents, exfoliant particles, texturing agents, optical brighteners, and insect repellants.

As examples of foaming and/or detergent surfactants, optionally present in the topical composition (C₂) according to the invention, mention may be made of anionic, cationic, amphoteric or nonionic foaming and/or detergent surfactants.

Among the anionic foaming and/or detergent surfactants that can be included in the topical composition (C₂) according to the invention, mention may be made of alkali metal salts, alkaline earth metal salts, ammonium salts, amine salts or amino alcohol salts of alkyl ether sulfates, alkyl sulfates, alkylamido ether sulfates, alkylarylpolyether sulfates, monoglyceride sulfates, alpha-olefin sulfonates, paraffin sulfonates, alkyl phosphates, alkyl ether phosphates, alkyl sulfonates, alkylamide sulfonates, alkylaryl sulfonates, alkyl carboxylates, alkyl sulfosuccinates, alkyl ether sulfosuccinates, alkylamide sulfosuccinates, alkyl sulfoacetates, alkylsarcosinates, acylisethionates, N-acyltaurates, acyl lactylates, N-acylated derivatives of amino acids, N-acylated derivatives of peptides, N-acylated derivatives of proteins, or N-acrylated derivatives of fatty acids.

Among the amphoteric foaming and/or detergent surfactants that can be included in the topical composition (C₂) according to the invention, mention may be made of alkyl betaines, alkylamido betaines, sultaines, alkylamidoalkyl sulfobetaines, imidazoline derivatives, phosphobetaines, amphopolyacetates and am phopropionates.

Among the cationic foaming and/or detergent surfactants that can be included in the topical composition (C₂) according to the invention, mention may particularly be made of quaternary ammonium derivatives.

Among the nonionic foaming and/or detergent surfactants that can be included in the topical composition (C₂) according to the invention, mention may more particularly be made of alkyl polyglycosides comprising a linear or branched, and saturated or unsaturated aliphatic radical, comprising from 8 to 16 carbon atoms, such as octylpolyglucoside, decylpolyglucoside, undecylenylpolyglucoside, dodecylpolyglucoside, tetradecylpolyglucoside, hexadecylpolyglucoside, 1,12-dodecanediylpolyglucoside; ethoxylated derivatives of hydrogenated castor oil, such as the product sold under the INCI name “Peg-40 hydrogenated castor oil”; polysorbates such as Polysorbate 20, Polysorbate 40, Polysorbate 60, Polysorbate 70, Polysorbate 80 or Polysorbate 85; coconut amides; N-alkylamines.

As examples of thickening and/or gelling surfactants optionally present in the topical composition (C₂) according to the invention, mention may be made of optionally alkoxylated alkyl polyglucoside fatty esters, for instance ethoxylated methylpolyglucoside esters such as PEG 120 methyl glucose trioleate and PEG 120 methyl glucose dioleate sold respectively under the names Glucamate™ LT and Glumate™ DOE120; alkoxylated fatty esters such as PEG 150 pentaerythrytyl tetrastearate sold under the name Crothix™ DS53, PEG 55 propylene glycol oleate sold under the name Antil™ 141; fatty-chain polyalkylene glycol carbamates, for instance PPG-14 laureth isophoryl dicarbamate sold under the name Elfacos™ T211, or PPG-14 palmeth-60 hexyl dicarbamate sold under the name Elfacos™ GT2125.

As examples of thickeners and/or gelling agents optionally present in the topical composition (C₂) according to the invention, mention may be made of linear or branched or crosslinked polymers of polyelectrolyte type, for instance acrylic acid homopolymer, methacrylic acid homopolymer, 2-methyl-[(1-oxo-2-propenyl)amino]-1-propane sulfonic acid (AMPS) homopolymer, copolymers of acrylic acid and of AMPS, copolymers of acrylamide and of AMPS, copolymers of vinylpyrolidone and of AMPS, copolymers of AMPS and of (2-hydroxyethyl) acrylate, copolymers of AMPS and of (2-hydroxyethyl) methacrylate, copolymers of AMPS and of hydroxyethylacrylamide, copolymers of AMPS and of N,N-dimethylacrylamide, copolymers of AMPS and of tris(hydroxy-methyl)acrylamidomethane (THAM), copolymers of acrylic or methacrylic acid and of (2-hydroxyethyl) acrylate, copolymers of acrylic or methacrylic acid and of (2-hydroxyethyl) methacrylate, copolymers of acrylic or methacrylic acid and of hydroxyethylacrylamide, copolymers of acrylic or methacrylic acid and of THAM, copolymers of acrylic or methacrylic acid and of N,N-dimethylacrylamide, terpolymers of acrylic or methacrylic acid, of AMPS and of (2-hydroxyethyl) acrylate, terpolymers of acrylic or methacrylic acid, of AMPS and of (2-hydroxyethyl) methacrylate, terpolymers of acrylic or methacrylic acid, of AMPS and of THAM, terpolymers of acrylic or methacrylic acid, of AMPS and of N,N-dimethylacrylamide, terpolymers of acrylic or methacrylic acid, of AMPS and of acrylamide, copolymers of acrylic acid or methacrylic acid and of alkyl acrylates of which the carbon-based chain comprises between four and thirty carbon atoms, and more particularly between ten and thirty carbon atoms, copolymers of AMPS and of alkyl acrylates of which the carbon-based chain comprises between four and thirty carbon atoms and more particularly between ten and thirty carbon atoms; the crosslinked polymers, and more particularly the crosslinked copolymers, terpolymers and tetrapolymers comprising AMPS and macromers, such as those described in the European patent applications published under numbers EP 1 069 142 A1 and EP 1 339 789 A2, in the international application published under number WO 2011/030044 Al and in the French patent application published under number 2 910 899 A1.

The linear or branched or crosslinked polymers of polyelectrolyte type optionally present in the topical composition (C₂) according to the invention may be in the form of a solution, an aqueous suspension, a water-in-oil emulsion, an oil-in-water emulsion, or a powder. The linear or branched or crosslinked polymers of polyelectrolyte type optionally present in the topical composition according to the invention can be selected from the products sold under the names Simulgel™ EG, Simulgel™ EPG, Sepigel™ 305, Simulgel™ 600, Simulgel™ NS, Simulgel™ INS 100, Simulgel™ FL, Simulgel™ A, Simulgel™ SMS 88, Sepinov™ EMT 10, Sepiplus™ 400, Sepiplus™ 265, Sepiplus™ S, Sepimax™ ZEN, Aristoflex™ HMB, Aristoflex™ VELVET, Aristoflex™ AVC, Aristoflex™ AVS, Novemer™ EC-1, Novemer™ EC-2, Flocare™ ET 25, Flocare™ ET 75, Flocare™ ET 26, Flocare™ ET 30, Flocare™ ET 58, Flocare™ PSD 30, Viscolam™ AT 64, Viscolam™ AT 100.

As examples of thickeners and/or gelling agents optionally present in the topical composition (C₂) according to the invention, mention may be made of polysaccharides consisting only of monosaccharides, such as glucans or glucose homopolymers, glucomannoglucans, xyloglycans, galactomannans of which the degree of substitution (DS) of the D-galactose units on the main chain of D-mannose is between 0 and 1, and more particularly between 1 and 0.25, such as galactomannans originating from cassia gum (DS=1/5), from locust bean gum (DS=1/4), from tara gum (DS=1/3), from guar gum (DS=1/2) or from fenugrec gum (DS=1).

As examples of thickeners and/or gelling agents optionally present in the topical composition (C₂) according to the invention, mention may be made of polysaccharides consisting of monosaccharide derivatives, such as sulfated galactans and more particularly carrageenans and agar, uronans and more particularly algins, alginates and pectins, heteropolymers of monosaccharides and of uronic acids and more particularly xanthan gum, gellan gum, exudates of gum Arabic and of karaya gum, and glucosaminoglycans.

As examples of thickeners and/or gelling agents optionally present in the topical composition (C₂) according to the invention, mention may be made of cellulose, cellulose derivatives, for example methylcellulose, ethylcellulose, hydroxypropylcellulose, silicates, starch, hydrophilic starch derivatives, and polyurethanes.

As examples of stabilizers optionally present in the topical composition (C₂) according to the invention, mention may for example be made of microcrystalline waxes, and more particularly ozokerite, mineral salts such as sodium chloride or magnesium chloride, and silicone polymers such as polysiloxane polyalkyl polyether copolymers.

As examples of solvents optionally present in the topical composition (C₂) according to the invention, mention may be made of water, organic solvents, for example glycerol, diglycerol, glycerol oligomers, ethylene glycol, propylene glycol, butylene glycol, 1,3-propanediol, 1,2-propanediol, hexylene glycol, diethylene glycol, xylitol, erythritol, sorbitol, water-soluble alcohols such as ethanol, isopropanol or butanol, and mixtures of water and of said organic solvents.

As examples of emulsifying surfactants optionally present in the topical composition (C₂) according to the invention, mention may be made of nonionic surfactants, anionic surfactants and cationic surfactants.

As examples of nonionic emulsifying surfactants optionally present in the topical composition (C₂) according to the invention, mention may be made of fatty acid esters of sorbitol, for instance the products sold under the names Montane™ 40, Montane™ 60, Montane™ 70, Montane™ 80 and Montane™ 85; compositions comprising glyceryl stearate and stearic acid ethoxylated at between 5 mol and 150 mol of ethylene oxide, for instance the composition comprising stearic acid ethoxylated at 135 mol of ethylene oxide and glyceryl stearate sold under the name Simulsol™ 165; mannitan esters; ethoxylated mannitan esters; sucrose esters; methylglucoside esters; alkyl polyglycosides comprising a linear or branched and saturated or unsaturated aliphatic radical, comprising from 14 to carbon atoms, such as tetradecylpolyglucoside, hexadecylpolyglucoside, octadecylpolyglucoside, hexadecylpolyxyloside, octadecylpolyxyloside, eicosylpolyglucoside, dodecosylpolyglucoside, 2-octyldodecylpolyxyloside or 12-hydroxystearylpolyglucoside; compositions of linear or branched and saturated or unsaturated fatty alcohols, comprising from 14 to 36 carbon atoms, and of alkyl polyglycosides as described above.

As examples of anionic surfactants optionally present in the topical composition (C₂) according to the invention, mention may be made of glyceryl stearate citrate, cetearyl sulfate, soaps, for example sodium stearate or triethanolammonium stearate, and N-acylated derivatives of amino acids which are salified, for example stearoyl glutamate.

As examples of cationic emulsifying surfactants optionally present in the topical composition (C₂) according to the invention, mention may be made of amine oxides, quaternium-82 and the surfactants described in the international application published under number WO 96/00719 and mainly those in which the fatty chain comprises at least 16 carbon atoms.

As examples of opacifiers and/or nacreous agents optionally present in the topical composition (C₂) according to the invention, mention may be made of sodium palmitate, sodium stearate, sodium hydroxystearate, magnesium palmitate, magnesium stearate, magnesium hydroxystearate, ethylene glycol monostearate, ethylene glycol distearate, polyethylene glycol monostearate, polyethylene glycol distearate, and fatty alcohols comprising from 12 to 22 carbon atoms.

As examples of texturing agents optionally present in the topical composition (C₂) according to the invention, mention may be made of N-acylated derivatives of fatty acids, for example the lauroyl lysine sold under the name Aminohope™ LL, the octenyl starch succinate sold under the name Dryflo™, the myristyl polyglucoside sold under the name Montanov™ 14, cellulose fibers, cotton fibers, chitosan fibers, talc, sericite and mica.

As examples of deodorants optionally present in the topical composition (C₂) according to the invention, mention may for example be made of alkali metal silicates, zinc salts such as zinc sulfate, zinc gluconate, zinc chloride or zinc lactate; quaternary ammonium salts such as cetyltrimethylammonium salts, cetylpyridinium salts; glycerol derivatives such as glyceryl caprate, glyceryl caprylate, polyglyceryl caprate; 1,2 decanediol; 1,3 propanediol; salicylic acid; sodium bicarbonate; cyclodextrins; metal zeolites; Triclosan™; aluminum bromohydrate, aluminum chlorohydrates, aluminum chloride, aluminum sulfate, aluminum zirconium chlorohydrates, aluminum zirconium trichlorohydrate, aluminum zirconium tetrachlorohydrate, aluminum zirconium pentachlorohydrate, aluminum zirconium octochlorohydrate, aluminum sulfate, sodium aluminum lactate, complexes of aluminum chlorohydrate and glycol, such as the complex of aluminum chlorohydrate and of propylene glycol, the complex of aluminum dichlorohydrate and of propylene glycol, the complex of aluminum sesquichlorohydrate and of propylene glycol, the complex of aluminum chlorohydrate and of polyethylene glycol, the complex of aluminum dichlorohydrate and of polyethylene glycol, and the complex of aluminum sesquichlorohydrate and of polyethylene glycol.

As examples of oils optionally present in the topical composition (C₂) according to the invention, mention may be made of mineral oils such as paraffin, liquid petroleum jelly, isoparaffins or white mineral oils; oils of animal origin, such as squalene or squalane; vegetable oils, such as phytosqualane, sweet almond oil, coconut oil, castor oil, jojoba oil, olive oil, rapeseed oil, peanut oil, sunflower oil, wheatgerm oil, corn germ oil, soybean oil, cottonseed oil, alfafa oil, poppyseed oil, pumpkin oil, evening primrose oil, millet oil, barley oil, rye oil, safflower oil, candle nut oil, passion flower oil, hazelnut oil, palm oil, shea butter, apricot kernel oil, betel leaf oil, sysymbrium oil, avocado oil, calendula oil, oils derived from flowers or from vegetables, ethoxylated vegetable oils; synthetic oils, for instance fatty acid esters such as butyl myristate, propyl myristate, isopropyl myristate, cetyl myristate, isopropyl palmitate, octyl palmitate, butyl stearate, hexadecyl stearate, isopropyl stearate, octyl stearate, isocetyl stearate, dodecyl oleate, hexyl laurate, propylene glycol dicaprylate, lanolic acid-derived esters, such as isopropyl lanolate, isocetyl lanolate, fatty acid monoglycerides, diglycerides and triglycerides, for instance glyceryl triheptanoate, alkyl benzoates, hydrogenated oils, poly(alpha-olefin)s, polyolefins, for instance poly(isobutene), synthetic isoalkanes, for instance isohexadecane, isododecane, perfluoro oils; silicone oils, for instance dimethylpolysiloxanes, methylphenylpolysiloxanes, silicones modified with amines, silicones modified with fatty acids, silicones modified with alcohols, silicones modified with alcohols and fatty acids, silicones modified with polyether groups, epoxy-modified silicones, silicones modified with fluoro groups, cyclic silicones and silicones modified with alcohol groups. The term “oils” is intended to mean, in the present application, compounds and/or mixtures of compounds which are insoluble in water and which have a liquid aspect at a temperature of 25° C.

As examples of waxes optionally present in the topical composition (C₂) according to the invention, mention may be made of beeswax, carnauba wax, candelilla wax, ouricury wax, Japan wax, cork fiber wax, sugarcane wax, paraffin wax, lignite waxes, microcrystalline waxes, lanolin wax; ozokerite; polyethylene wax; silicone waxes; vegetable waxes; fatty alcohols and fatty acids which are solid at ambient temperature; glycerides which are solid at ambient temperature. The term “waxes” is intended to mean, in the present application, compounds and/or mixtures of compounds which are insoluble in water and which have a solid aspect at a temperature greater than or equal to 45° C.

As examples of active ingredients optionally present in the topical composition (C₂) according to the invention, mention may be made of vitamins and derivatives thereof, in particular esters thereof, such as retinol (vitamin A) and esters thereof (retinyl palmitate for example), ascorbic acid (vitamin C) and esters thereof, sugar derivatives of ascorbic acid (for instance ascorbyl glucoside), and esters thereof (for instance tocopheryl acetate), vitamin B3 or B10 (niacinamide and derivatives thereof); compounds showing a skin-lightening or skin-depigmenting action, for instance the ω-undecelynoyl phenylalanine sold under the name Sepiwhite™ MSH, Sepicalm™ VG, the glycerol monoester and/or diester of ω-undecelynoyl phenylalanine, ω-undecelynoyl dipeptides, arbutin, kojic acid, hydroquinone; compounds showing a soothing action, in particular Sepicalm™ S, allantoin and bisabolol; anti-inflammatories; compounds showing a moisturizing action, such as urea, hydroxyureas, glycerol, polyglycerols, glycerolglucoside, diglycerolglucoside, polyglycerylglucosides, xylitylglucoside; polyphenol-rich plant extracts, for instance grape extracts, pine extracts, wine extracts, olive extracts; compounds showing a slimming or lipolytic action, for instance caffeine or derivatives thereof, Adiposlim™, Adipoless™ fucoxanthine; N-acylated proteins; N-acylated peptides, for instance Matrixil™; N-acylated amino acids; partial hydrolysates of N-acylated proteins; amino acids; peptides; total protein hydrolysates; soybean extracts, for example Raffermine™; wheat extracts, for example Tensine™ or Gliadine™; plant extracts, such as tannin-rich plant extracts, isoflavone-rich plant extracts or terpine-rich plant extracts; extracts of freshwater or seawater algae; marine plant extracts; marine extracts in general, such as corals; essential waxes; bacterial extracts; ceramide; phospholipids; compounds showing an antimicrobial action or a purifying action, for instance Lipacide™ C8G, Lipacide™ UG, Sepicontrol™ A5; Octopirox™ or Sensiva™ SC50; compounds showing an energizing or stimulating property, for instance Physiogenyl™, panthenol and derivatives thereof, for instance Sepicap™ MP; anti-aging active agents, for instance Sepilift™ DPHP, Lipacide™ PVB, Sepivonol™, Sepivital™, Manoliva™, Phyto-Age™, Timecode™; Survicode™; anti-photoaging active agents; agents for protecting the integrity of the dermoepidermal junction; active agents for increasing the synthesis of extracellular matrix components such as collagen, elastins, glycosaminoglycans; active agents which act favorably on chemical cell communication, for instance cytokines, or physical cell communication, for instance integrins; active agents which create a “heating” sensation on the skin, for instance skin microcirculation activators (for instance nicotinic acid derivatives) or products which create a feeling of “freshness” on the skin (for instance methanol and derivatives); active agents for improving skin microcirculation, for example veinotonics; draining active agents; active agents for decongestive purposes, for instance extracts of ginko biloba, of ivy, of horse chestnut, of bamboo, of ruscus, of butcher's broom, of Centalla asiatica, of fucus, of rosemary, of willow; agents for tanning or browning the skin, for instance dihydroxyacetone, isatin, alloxane, ninhydrin, glyceraldehyde, mesotartic aldehyde, glutaraldehyde, erythrulose.

As examples of sunscreens optionally present in the topical composition (C₂) according to the invention, mention may be made of all those which appear in modified cosmetics directive 76/768/EEC annex VII.

Among the organic sunscreens optionally present in the topical composition (C₂) according to the invention, mention may be made of the family of benzoic acid derivatives, for instance para-aminobenzoic acids (PABAs), in particular monoglyceryl esters of PABA, ethyl esters of N,N-propoxy PABA, ethyl esters of N,N-diethoxy PABA, ethyl esters of N,N-dimethyl PABA, methyl esters of N,N-dimethyl PABA, butyl esters of N,N-dimethyl PABA; the family of anthranilic acid derivatives, for instance homomenthyl-N-acetyl anthranilate; the family of salicylic acid derivatives, for instance amyl salicylate, homomenthyl salicylate, ethylhexyl salicylate, phenyl salicylate, benzyl salicylate, or p-isopropanolphenyl salicylate; the family of cinnamic acid derivatives, for instance ethylhexyl cinnamate, ethyl-4-isopropyl cinnamate, methyl 2,5-diisopropylcinnamate, propyl 4-methoxycinnamate, isopropyl 4-methoxycinnamate, isoamyl 4-methoxy-cinnamate, octyl 4-methoxycinnamate (2-ethylhexyl 4-methoxycinnamate), 2-ethoxyethyl 4-methoxycinnamate, cyclohexyl 4-methoxycinnamate, ethyl α-cyano-β-phenylcinnamate, 2-ethylhexyl α-cyano-β-phenylcinnamate, mono(2-ethylhexanoyl)glyceryl di(paramethoxy-cinnamate); the family of benzophenone derivatives, for instance 2,4-dihydroxy-benzophenone, 2,2′-dihydroxy-4-methoxybenzophenone, 2,2′,4,4′-tetrahydroxybenzo-phenone, 2-hydroxy-4-methoxybenzophenone, 2-hydroxy-4-methoxy-4′-methylbenzo-phenone, 2-hydroxy-4-methoxybenzophenone-5-sulfonate, 4-phenylbenzophenone, 2-ethylhexyl-4′-phenylbenzophenone-2-carboxylate, 2-hydroxy-4-n-octyloxybenzophe-none, 4-hydroxy-3-carboxybenzophenone; 3-(4′-methyl benzylidene)-d,l-camphor, 3-(benzylidene)-d,l-camphor, camphor benzalkonium methosulfate; urocanic acid, ethyl urocanate; the family of sulfonic acid derivatives, for instance 2-phenylbenzimidazole-5 sulfonic acid and salts thereof; the family of triazine derivatives, for instance hydroxyphenyl triazine, ethylhexyloxyhydroxyphenyl-4-methoxyphenyltriazine, 2,4,6-trianillino-(p-carbo-2′-ethylhexyl-1′-oxy)-1,3,5-triazine, benzoic acid 4,4-((6-(((1,1-dimethylethyl)amino)-carbonyl)phenyl)amino)-1,3,5-triazine-2,4-diyl diimino) bis(2-ethylhexyl) ester, 2-phenyl-5-methylbenzoxazole, 2,2′-hydroxy-5-methylphenylbenzotriazole, 2-(2′-hydroxy-5′-t-octyl-phenyl)benzotriazole, 2-(2′-hydroxy-5′-methyphenyl)benzotriazole; dibenzazine; dianisoylmethane, 4-methoxy-4″-t-butylbenzoylmethane; 5-(3,3-dimethyl-2-norbornylidene)-3-pentan-2-one; the family of diphenyl acrylate derivatives, for instance 2-ethylhexyl-2-cyano-3,3-diphenyl-2-propenoate, ethyl-2-cyano-3,3-diphenyl-2-propenoate; the family of polysiloxanes, for instance benzylidene siloxane malonate.

Among the inorganic sunscreens, also called “mineral screens”, optionally present in the topical composition (C₂) according to the invention, mention may be made of titanium oxides, zinc oxides, cerium oxide, zirconium oxide, yellow, red or black iron oxides, and chromium oxides. These mineral screens may or may not be micronized, may or may not have undergone surface treatments and may be optionally provided in the form of aqueous or oily predispersions.

The topical composition (C₂) according to the invention may be more particularly a suspension of solid particles. Said suspended solid particles present in the topical composition (C₂) according to the invention may have various regular or irregular geometries, and may be in the form of beads, balls, webs, flakes, lamellae or polyhedra. These solid particles are characterized by an apparent average diameter of between 1 micrometer and 5 millimeters, more particularly between 10 micrometers and 1 millimeter. Among the solid particles, mention may be made of micas, iron oxide, titanium oxide, zinc oxide, aluminum oxide, talc, silica, kaolin, clays, boron nitride, calcium carbonate, magnesium carbonate, magnesium hydrogen carbonate, inorganic colored pigments, polyamides such as nylon-6, polyethylenes, polypropylenes, polystyrenes, polyesters, acrylic or methacrylic polymers such as poly(methyl methacrylate)s, polytetrafluoro-ethylene, crystalline or microcrystalline waxes, porous spheres, selenium sulfide, zinc pyrithione, starches, alginates, vegetable fibers, Loofah particles and sponge particles.

According to another aspect, a subject of the invention is a composition (C₂) as defined above, for carrying out a method of therapeutic treatment of the human or animal body, intended for treating and/or preventing redness and/or irritations visible on the skin and/or the scalp due to solar radiation.

The composition (C₂) as defined above may for example be administered topically.

The following experimental study illustrates the invention without, however, limiting it.

1) Preparation of Compositions Represented by Formula (I) and Evaluation of their Solubilizing Properties According to the Invention.
1.1 Preparation of a composition of n-heptyl polyglucosides

2.7 molar equivalents of n-heptanol are introduced into a jacketed glass reactor, in which a heat-transfer fluid circulates, and which is equipped with efficient stirring, at a temperature of 40° C. One molar equivalent of anhydrous glucose is then gradually added to the reaction medium so as to allow its uniform dispersion, then 0.15% by weight of 98% sulfuric acid and 0.15% by weight of 50% hypophosphorous acid, for 100% of the weight made up of the sum of the weight of glucose and of the weight of n-heptanol, are introduced into the previously prepared uniform dispersion. The reaction medium is placed under a partial vacuum of approximately 180 mbar, and maintained at a temperature of 100° C.-105° C. for a period of 4 hours with evacuation of the water formed by means of a distillation assembly. The reaction medium is then cooled to 85° C.-90° C. and neutralized by adding 40% sodium hydroxide, so as to bring the pH of a 5% solution of this mixture to a value of approximately 7.0. The reaction medium thus obtained is then emptied out at a temperature of 70° C. and filtered to remove the unreacted glucose grains. The filtrate is then introduced into a jacketed glass reactor, in which a heat-transfer fluid circulates, and which is equipped with efficient stirring and with a distillation device. The excess heptanol is then removed by distillation at a temperature of 120° C. under a partial vacuum of between approximately 100 mbar and 50 mbar. The reaction medium thus distilled is immediately diluted by adding an amount of water so as to achieve a concentration of reaction medium of approximately 60%. After homogenization, for 30 minutes at a temperature of 50° C., the composition (X₁) obtained is emptied out.

The analytical characteristics of the composition (X₁) thus obtained comprising n-heptyl polyglucosides are collated in table 1 below.

TABLE 1
Analytical characteristics of the composition (X₁).

| | Compo- |
|---|---|
| | sition (X₁) |
| Appearance at 20° C. (visual determination) | Liquid |
| Acid number (standard NFT 60204) | 1.7 |
| Hydroxyl number on dry extract (standard USP XXI NF | 813.9 |
| XVI 01/01/1995) | |
| Water (% by weight) (standard NFT 73201) | 58.8% |
| Residual content of n-heptanol (gas chromatography) as | 0.22% |
| % by weight | |

1.2 Evaluation of the vitamin E solubilizing properties of n-heptyl polyglucosides, of 2-ethylhexyl polyglucosides and of n-butyl polyglucosides in water

The solubilizing properties of the composition (X₁) of n-heptyl polyglucosides, obtained according to the process previously described, of the composition of (2-ethyl-hexyl)polyglucosides, sold under the trade name AG 6202 by the company Akzo Nobel (composition (X₂)), and of the composition of n-butyl polyglucosides, sold under the trade name Simulsol™ SL4 (composition (X₄)), were evaluated in comparison with those of a composition of hydrogenated castor oil ethoxylated at 40 mol of ethylene oxide (composition (X₃)), the INCI name of which is “PEG-40 hydrogenated castor oil” and the CAS number of which is 61788-85-0, sold under the name Simulsol™ 1293 by the company SEPPIC, representing the prior art, according to the evaluation method described below for DL-α-tocopherol.

1.2.1-Principle of the method of evaluating the solubilization, in an aqueous medium, of DL-α-tocopherol by the compositions according to the invention and by the composition according to the prior art

The object of this method is to determine the solubilizing capacity of surfactant compositions, (X₁) and (X₂), in an aqueous medium, for DL-α-tocopherol which is insoluble in an aqueous medium, compared with surfactant compositions of the prior art (X₃) and (X₄).

### 1.2.2-Experimental Protocol

For each composition (X₁), (X₂), (X₃) and (X₄) to be evaluated, an amount of 10 grams of solids of said composition to be evaluated are introduced into a 150 cm³ glass flask. A predetermined weight fraction of DL-α-tocopherol to be solubilized is then introduced. An amount of distilled water is additionally added to as to obtain a weight of 100 grams. The temperature is regulated at 20° C. or 45° C. A magnetized magnetic bar is introduced into the glass flask, which is then placed under magnetic stirring at a speed of 80 revolutions/min for a period of 60 minutes at a temperature of 20° C. or of 45° C. At the end of this period, the visual appearance obtained is noted. If the mixture is clear, another test is carried out under the operating conditions described above with a higher weight fraction of DL-α-tocopherol; if the mixture is cloudy and not clear, another test is carried out under the operating conditions described above with a smaller weight fraction of DL-α-tocopherol.

### 1.2.3-Expression of the Results

The visual appearance of the solution obtained according to the protocol of section 1.2.2 of the present application is noted by the experimenter and described as “clear” or as “cloudy” as appropriate.

1.2.4-Characterization of the solubilization in an aqueous medium, at a temperature of 20° C., of DL-α-tocopherol by the compositions (X₁) and (X₂) comprising the compounds of formula (I) according to the invention and by the compositions (X₃) and (X₄) of the prior art

The experimental protocol described in section 1.2.2 of the present application was carried out at a temperature of 20° C. for the compositions (X₁) and (X₂) according to the invention and for the compositions (X₃) and (X₄) of the prior art.

The appearances of the solutions prepared by carrying out the operating protocol for each of the compositions (X₁), (X₂), (X₃) and (X₄) used to solubilize the DL-α-tocopherol at 20° C. were recorded by the experimenter and noted respectively in tables 2, 3, 4 and 5 below.

TABLE 2
Appearance of solutions comprising the nonionic surfactant composition
(X₁) and a variable amount of DL-α-tocopherol at 20° C.

| Weight fraction of DL-α-tocopherol | Appearance of the solutions |
|---|---|
| 0% | Clear |
| 0.1% | Clear |
| 0.5% | Clear |
| 1.0% | Clear |
| 2.0% | Clear |
| 4.0% | Clear |
| 5.0% | Clear |
| 6.0% | Clear |
| 6.5% | Clear |
| 6.7% | Cloudy |
| 7.0% | Cloudy |

TABLE 3
Appearance of solutions comprising the nonionic surfactant composition
(X₂) and a variable amount of DL-α-tocopherol at 20° C.

| Weight fraction of DL-α-tocopherol | Appearance of the solutions |
|---|---|
| 0% | Clear |
| 0.1% | Clear |
| 0.5% | Clear |
| 1.0% | Clear |
| 2.0% | Clear |
| 4.0% | Clear |
| 4.1% | Clear |
| 4.2% | Cloudy |
| 4.5% | Cloudy |
| 5.0% | Cloudy |

TABLE 4
Appearance of solutions comprising the nonionic surfactant composition
(X₃) and a variable amount of DL-α-tocopherol at 20° C.

| Weight fraction of DL-α-tocopherol | Appearance of the solutions |
|---|---|
| 0% | Clear |
| 0.1% | Cloudy |
| 0.5% | Cloudy |
| 1.0% | Cloudy |

TABLE 5
Appearance of solutions comprising the nonionic surfactant composition
(X₄) and a variable amount of DL-α-tocopherol at 20° C.

| Weight fraction of DL-α-tocopherol | Appearance of the solutions |
|---|---|
| 0% | Clear |
| 0.5% | Cloudy |
| 1.5% | Cloudy |
| 3.0% | Cloudy |
| 5.0% | Cloudy |
| 6.5% | Cloudy |

1.2.5-Characterization of the solubilization in an aqueous medium, at a temperature of 45° C., of DL-α-tocopherol by the compositions (X₁) and (X₂) comprising the compounds of formula (I) according to the invention and by the composition (X₃) of the prior art

The experimental protocol described in section 1.2.2 of the present application was carried out at a temperature of 45° C. for the compositions (X₁) and (X₂) according to the invention and for the composition (X₃) of the prior art.

The appearances of the solutions prepared by carrying out the operating protocol for each of the compositions (X₁), (X₂) and (X₃) used to solubilize the DL-α-tocopherol at 45° C. were recorded by the experimenter and noted respectively in tables 6, 7 and 8 below.

TABLE 6
Appearance of solutions comprising the nonionic surfactant composition
(X₁) and a variable amount of DL-α-tocopherol at 45° C.

| Weight fraction of DL-α-tocopherol | Appearance of the solutions |
|---|---|
| 0% | Clear |
| 1.0% | Clear |
| 2.0% | Clear |
| 4.0% | Clear |
| 5.0% | Clear |
| 6.0% | Clear |
| 6.5% | Clear |
| 6.7% | Cloudy |
| 7.0% | Cloudy |

TABLE 7
Appearance of solutions comprising the nonionic surfactant composition
(X₂) and a variable amount of DL-α-tocopherol at 45° C.

| Weight fraction of DL-α-tocopherol | Appearance of the solutions |
|---|---|
| 0% | Clear |
| 1.0% | Clear |
| 2.0% | Clear |
| 4.0% | Clear |
| 4.1% | Clear |
| 4.2% | Cloudy |
| 4.5% | Cloudy |
| 5.0% | Cloudy |

TABLE 8
Appearance of solutions comprising the nonionic surfactant composition
(X₃) and a variable amount of DL-α-tocopherol at 45° C.

| Weight fraction of DL-α-tocopherol | Appearance of the solutions |
|---|---|
| 0% | Clear |
| 0.1% | Clear |
| 0.5% | Cloudy |
| 1.0% | Cloudy |

### 1.2.6 Analysis of the Results

The compositions (X₁) and (X₂), comprising respectively n-heptyl polyglucosides and (2-ethylhexyl) polyglucosides, make it possible to solubilize in water respective weight proportions of 6.5% and 4.1% of DL-α-tocopherol at a temperature of 20° C., whereas, under the same operating conditions, the comparative compositions (X₃) and (X₄) do not make it possible to solubilize DL-α-tocopherol in water.

The compositions (X₁) and (X₂), comprising respectively n-heptyl polyglucosides and (2-ethylhexyl) polyglucosides, are characterized by the same capacities for solubilizing DL-α-tocopherol in water at an operating temperature of 45° C.

1.2.7 Stability of the solutions of DL-α-tocopherol in the presence of the compositions (X₁) and (X₂)

a) Two solutions each comprising 10% by weight of solids of the composition (X₁) and weight contents of DL-α-tocopherol respectively of 6.0% and of 6.5% were stored for 7 days without stirring, in an air-conditioned chamber regulated at a temperature of 20° C. At the end of this period, each of the solutions has a clear appearance.

b) Two solutions each comprising 10% by weight of solids of the composition (X₂) and weight contents of DL-α-tocopherol respectively of 3.0% and of 4.0%, were stored for 7 days without stirring, in an air-conditioned chamber regulated at a temperature of 20° C. At the end of this period, each of the solutions has a clear appearance.

c) Two solutions each comprising 10% by weight of solids of the composition (X₁) and weight contents of DL-α-tocopherol respectively of 6.0% and of 6.5% were stored for 7 days without stirring, in an air-conditioned chamber regulated at a temperature of 45° C. At the end of this period, each of the solutions has a clear appearance.

d) Two solutions each comprising 10% by weight of solids of the composition (X₂) and weight contents of DL-α-tocopherol respectively of 3.0% and of 4.0% were stored for 7 days without stirring, in an air-conditioned chamber regulated at a temperature of 45° C. At the end of this period, each of the solutions has a clear appearance.

### 1.2.7 Conclusions

The compositions (X₁) and (X₂) comprising respectively n-heptyl polyglucosides and (2-ethylhexyl) polyglucosides make it possible to stably solubilize DL-α-tocopherol compared with the solubilizing agents known in the prior art.

1. A composition comprising:
a composition represented by formula (I):
R—O-(G)ₚ-H (I)
in which R represents a radical chosen from n-hexyl, n-heptyl or 2-ethylhexyl radicals, G represents the residue of a reducing sugar and p represents a decimal number greater than or equal to 1.05 and less than or equal to 5, said composition of formula (I) consisting of a mixture of compounds represented by formulae (I₁), (I₂), (I₃), (I₄) and (I₅):
R—O-(G)₁-H (I₁)
R—O-(G)₂-H (I₂)
R—O-(G)₃-H (I₃)
R—O-(G)₄-H (I₄)
R—O-(G)₅-H (I₅)
in the respective molar proportions a₁, a₂, a₃, a₄ and a₅, such that:
the sum a₁+a₂+a₃+a₄+a₅ is equal to 1 and that
each of the proportions a₁, a₂, a₃, a₄ and a₅ is greater than or equal to zero and less than or equal to one; and
at least one compound (A) chosen from α-tocopherol, β-tocopherol, γ-tocopherol, δ-tocopherol, α-tocopheryl acetate, β-tocopheryl acetate, γ-tocopheryl acetate, δ-tocopheryl acetate, α-tocotrienol, β-tocotrienol, γ-tocotrienol and δ-tocotrienol.

2. The aqueous composition as defined in claim 1, wherein, in formula (I), p represents a decimal number greater than or equal to 1.05 and less than or equal to 2.5.

3. The aqueous composition as defined in claim 1, wherein, in formula (I), G represents the residue of a reducing sugar chosen from the residues of glucose, of xylose and of arabinose.

4. The aqueous composition as defined in claim 1, wherein the compound (A) is α-tocopherol or α-tocopheryl acetate.

5. The aqueous composition as defined in claim 1, wherein the weight ratio between the compound (A) and the composition represented by formula (I) is greater than or equal to 1/10 and less than or equal to 6.5/10.

6. A composition (C₂) comprising, for 100% of its weight:
a) from 0.05% to 12.0% by weight of at least one composition represented by formula (I): as dcfincd in claim 1
R—O-(G)ₚ-H (I)
in which R represents a radical chosen from n-hexyl, n-heptyl or 2-ethylhexyl radicals, G represents the residue of a reducing sugar and p represents a decimal number greater than or equal to 1.05 and less than or equal to 5, said composition of formula (I) consisting of a mixture of compounds represented by formulae (I₁), (I₂), (I₃), (I₄) and (I₅):
R—O-(G)₁-H (I₁)
R—O-(G)₂-H (I₂)
R—O-(G)₃-H (I₃)
R—O-(G)₄-H (I₄)
R—O-(G)₅-H (I₅)
in the respective molar proportions a₁, a₂, a₃, a₄ and a₅, such that:
the sum a₁+a₂+a₃+a₄+a₅ is equal to 1 and that
each of the proportions a₁, a₂, a₃, a₄ and a₅ is greater than or equal to zero and less than or equal to one;
b) from 0.01 to 5.0% by weight of at least one compound (A) chosen from α-tocopherol, β-tocopherol, γ-tocopherol, δ-tocopherol, α-tocopheryl acetate, β-tocopheryl acetate, γ-tocopheryl acetate, δ-tocopheryl acetate, α-tocotrienol, β-tocotrienol, γ-tocotrienol and δ-tocotrienol;
c) from 83.0% to 99.94% by weight of water.

7. The composition (C₂) as defined in claim 6, wherein the compound (A) is α-tocopherol or α-tocopheryl acetate.

8. The composition (C₂) as defined in claim 1, wherein the weight ratio between the compound (A) and the composition represented by formula (I) is greater than or equal to 1/10 and less than or equal to 6.5/10.

9. A method for preventing the unattractive effects of aging of the skin, the hair, the scalp or the mucous membranes, comprising administering to a subject in need thereof an effective amount of the composition (C₂) as defined in claim 6.

10. A process for preparing a composition intended for cleansing the skin, the hair, the scalp or the mucous membranes, comprising combining the composition (C₂) as defined in claim 6 with other components of a composition intended for cleansing the skin, the hair, the scalp or the mucous membranes.

11. A method of therapeutically treating a human or animal body, intended for treating and/or preventing redness and/or irritations visible on the skin and/or the scalp due to solar radiation, comprising administering to a human or animal body in need thereof a therapeutically effective amount of the composition (C₂) as defined in claim 6.
